(19) United States
(12) Patent Application Publication
(10) Pub. No.: US 2011/0275882 A1
(43) Pub. Date: Nov. 10, 2011

(54) VALVE FOR VENTRICULAR ASSIST DEVICE

(75) Inventors: JENS HUTZENLAUB, Aachen (DE); Michael Gaul, Tucson, AZ (US)

(73) Assignee: SYNCARDIA SYSTEMS, INC, Tucson, AZ (US)

(21) Appl. No.: 12/774,103

(22) Filed: May 5, 2010

(51) Int. Cl. A61M 1/10; F16K 1/18

(52) U.S. Cl. 600/16; 251/301

(57) ABSTRACT

A ventricular-assist-device valve includes two flaps hinged to a peripheral wall by means of a couple of flexible struts. As each flap opens and closes during each cycle of operation, the struts flex and open passages for flow around them, around the flaps, and between the struts and the flaps, so as to prevent the formation of dead zones that contribute to the accumulation and deterioration of blood cells that produce clotting. The struts are tensioned so as to exert a pressure against the flow of the blood stream when the flaps are open. This tension creates a pressure differential between the underside and the peripheral regions of the flaps that forces blood flow around the hinges and washes out stagnant cells. As a result, clotting is materially reduced.

### BACKGROUND OF THE INVENTION

1. Field of the Invention

The invention relates generally to artificial heart pumps and, in particular, to an improved valve for reducing thrombus in ventricular assist devices.

2. Description of the Prior Art

The heart is the muscle that drives the cardiovascular system in living beings. Acting as a pump, the heart moves blood throughout the body to provide oxygen, nutrients, hormones, and to remove waste products. The blood follows two separate pathways in the human body, the so-called pulmonary and systemic circulatory circuits. In the pulmonary circuit, the heart pumps blood first to the lungs to release carbon dioxide and bind oxygen, and then back to the heart. Thus, oxygenated blood is constantly being supplied to the heart. In the systemic circuit, the longer of the two, the heart pumps oxygenated blood through the rest of the body to supply oxygen and remove carbon dioxide, the byproduct of metabolic functions carried out throughout the body. The heart supplies blood to the two circuits with pulses generated by the orderly muscular contraction of its walls.

In order to keep blood moving through these two separate circulatory circuits, the human heart has four distinct chambers that work in pairs. As illustrated in FIG. 1, the heart 10 includes a right atrium 12, a right ventricle 14, a left atrium 16, and a left ventricle 18. One pair of chambers, the right ventricle and left atrium, is connected directly to the pulmonary circuit. In it, de-oxygenated blood from the body is pumped from the right ventricle 14 to the lungs, where it is oxygenated, and then back to the left atrium 16.

In the systemic circuit, the other pair of chambers pumps the oxygenated blood through body organs, tissues and bones. The blood moves from the left atrium 16, where it flows from the lungs, to the left ventricle 18, which in turn pumps the blood throughout the body and all the way back to the right atrium 12. The blood then moves to the right ventricle 14 where the cycle is repeated. In each circuit, the blood enters the heart through an atrium and leaves the heart through a ventricle.

Thus, the ventricles 14,18 are essentially two separate pumps that work together to move the blood through the two circulatory circuits. Four check valves control the flow of blood within the heart and prevent flow in the wrong direction. A tricuspid valve 20 controls the blood flowing from the right atrium 12 into the right ventricle 14. Similarly, a bicuspid valve 22 controls the blood flowing from the left atrium 16 into the left ventricle 18. Two semilunar valves (pulmonary 24 and aortic 26) control the blood flow leaving the heart toward the pulmonary and systemic circuits, respectively. Thus, in each complete cycle, the blood is pumped by the right ventricle 14 through the pulmonary semilunar valve 24 to the lungs and back to the left atrium 16. The blood then flows through the bicuspid valve 22 to the left ventricle 18, which in turn pumps it through the aortic semilunar valve 26 throughout the body and back to the right atrium 12. Finally, the blood flows back to the right ventricle 14 through the tricuspid valve 20 and the cycle is repeated.

When the heart muscle squeezes each ventricle, it acts as a pump that exerts pressure on the blood, thereby pushing it out of the heart and through the body. The blood pressure, an indicator of heart function, is measured when the heart muscle contracts as well as when it relaxes. The so-called systolic pressure is the maximum pressure exerted by the blood on the arterial walls when the left ventricle of the heart contracts forcing blood through the arteries in the systemic circulatory circuit. The so-called diastolic pressure is the lowest pressure on the blood vessel walls when the left ventricle relaxes and refills with blood. Healthy blood pressure is considered to be about 120 millimeters of mercury systolic and 80 millimeters of mercury diastolic (usually presented as 120/80).

Inasmuch as the function of the circulatory system is to service the biological needs of all body tissues (i.e., to transport nutrients to the tissues, transport waste products away, distribute hormones from one part of the body to another, and, in general, to maintain an appropriate environment for optimal function and survival of tissue cells), the rate at which blood is circulated by the heart is a critical aspect of its function. The heart has a built-in mechanism (the so-called Frank-Starling mechanism) that allows it to pump automatically whatever amount of blood flows into it. Such cardiac output in a healthy human body may vary from about 4 to about 15 liters per minute (LPM), according to the activity being undertaken by the person, at a heart rate that can vary from about 50 to about 180 beats per minute.

Several artificial devices have been developed over the years to supplement or replace the function of a failing heart in a patient. Typically, these artificial devices consist of pumps that aim at duplicating the required pumping functions of the left and right human ventricles. Ventricular assist devices, normally referred to as VADs, are mechanical circulatory devices used to partially or completely replace the function of a failing heart. Some VADs are used for a short term in patients recovering from heart attacks or heart surgery, while others are used for months or even years in patients suffering from congestive heart failure.

In contrast to artificial hearts, which are designed to completely take over the cardiac function and generally require the removal of the patient's heart, VADs are designed to assist either the left or the right ventricle, or both. They are either implanted or connected externally between the left ventricle and the aorta or the right ventricle and the pulmonary artery, respectively. Left ventricle VADs are most commonly used, but right ventricular assistance may become necessary as well when pulmonary arterial resistance is high. Long-term VADs are normally used to keep patients alive with a good quality of life while they wait for a heart transplant. However, VADs are sometimes also used in therapeutic applications and as a bridge to recovery.

Most VADs utilize two valves connected to a pump. One valve controls the inflow to the pump chamber, while the other controls its outflow into the patient's circulatory system. Therefore, these valves are critical to the operation of the VAD and the survival of the patient. Over the years, these valves have consisted either of bioprostheses made of animal heart valves or tissue, or of mechanical valves made of plastic materials. Bioprostheses exhibit high biocompatibility but are not suitable for long-term applications because of their limited durability. Mechanical valves are durable but produce blood clotting (“thrombus”) because of the blood flowing over a non-biological surface. This is a recurring problem in the performance of VADs and anticoagulant compounds are typically used to reduce the risk of malfunction. However, clotting remains the most serious hurdle for the long-term use of mechanical VADS in patients.

The design of mechanical VAD valves has evolved over time with the dual objectives of improving durability, which of course is the most critical aspect of VADs' performance, and of minimizing thrombus. The geometry of the valve, in addition to the material, is believed to be most crucial for minimizing thrombus. Early ball-valve designs were replaced by valves with disk-shaped flaps sealing the circular passage in and out of the VAD. Various geometries have been implemented with one, two or three flaps hinged to a peripheral ring, but none has produced a satisfactory solution to the clotting problem. The relatively rough closing mechanisms and the dead-flow zones around the hinges of the flaps are the source of clotting in these valves. Therefore, the present invention involves a novel flap design directed at producing a smoother closing motion and at eliminating areas of blood accumulation within the valve, especially around the points of attachment of the flaps.

### SUMMARY OF THE INVENTION

A major concern in designing an improved valve for ventricular assist devices is a configuration that reduces turbulence and promotes flow around all components of the valve so as to eliminate dead zones that increase the chance of clotting. To that end, according to one aspect of the invention, the new VAD valve includes multiple flaps, preferably two, hinged to a peripheral wall by means of a couple of flexible struts. As each flap opens and closes during each cycle of operation, the struts flex and open passages for flow around them, around the flaps, and between the struts and the flaps, so as to prevent the formation of dead zones that contribute to the accumulation and deterioration of blood cells that produce clotting.

According to another, very important, aspect of the invention, the struts are tensioned so as to exert a pressure against the flow of the blood stream when the flaps are open. This tension creates a pressure differential between the underside and the peripheral regions of the flaps that forces blood flow around the hinges and washes out any stagnant cells. As a result, clotting is reduced materially in comparison with prior-art valves.

Additional features and advantages of the invention will be forthcoming from the following detailed description of certain specific embodiments when read in conjunction with the accompanying drawings.

### BRIEF DESCRIPTION OF THE DRAWINGS

FIG. 1 is a representation of the human heart.

FIG. 2 is a schematic elevational view of a valve according to the present invention in closed position.

FIG. 3 is a top view corresponding to FIG. 2.

FIG. 4 is a schematic elevational view of the valve of the invention in fully open position.

FIG. 5 is a top view corresponding to FIG. 4.

FIG. 6 is a schematic elevational view of the valve of the invention in partially open position.

FIG. 7 is a top view corresponding to FIG. 6.

FIG. 8 shows three perspective views of the valve of the invention during a cycle of operation.

FIG. 9 is a partially cut-out elevational view of the valve of the invention showing a front view of a flap in open position.

FIG. 10 illustrates the valves of the invention installed in a conventional diaphragm-actuated ventricular assist device.

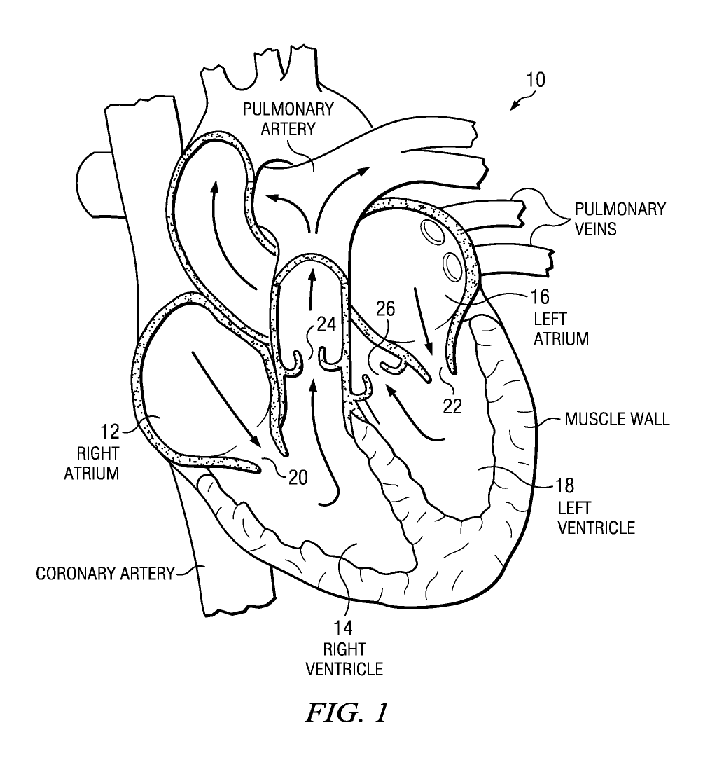

### DESCRIPTION OF THE PREFERRED EMBODIMENTS

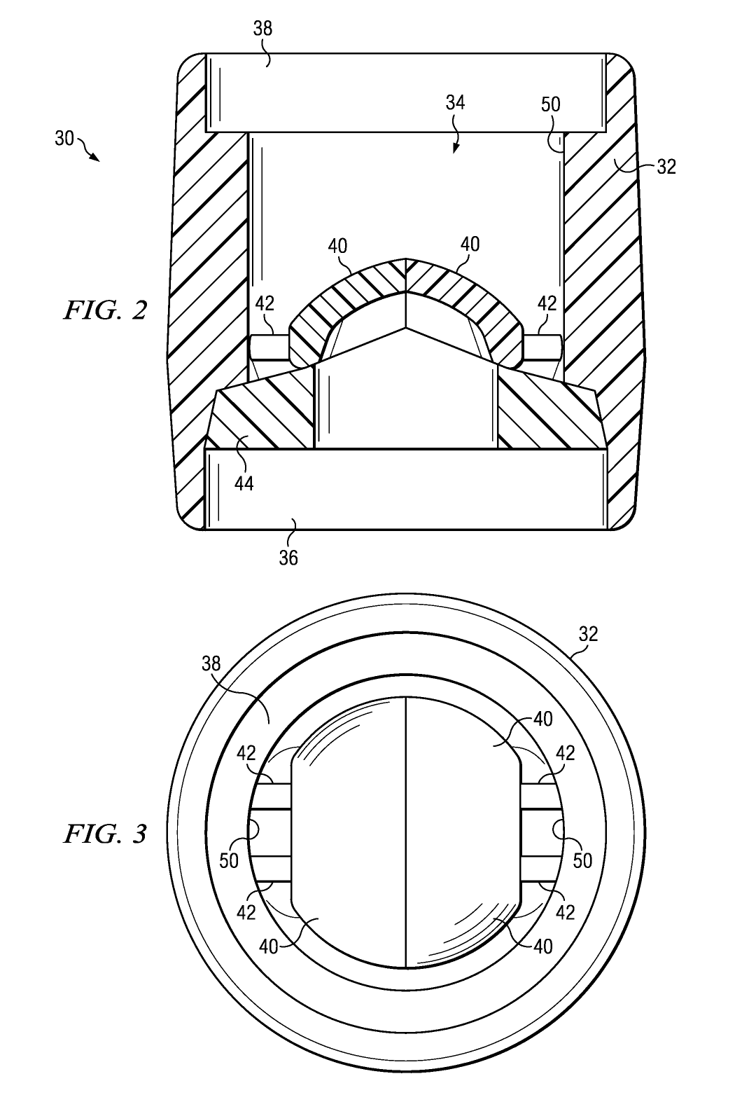

Referring to the figures, wherein the same reference numerals and symbols are used to refer to equal parts, FIG. 2 shows a valve 30 according to the present invention. The valve includes a housing 32 adapted for connection to the pump chamber of a ventricular assist device (not shown). The housing 32 defines a passage 34, normally cylindrical in configuration, for flowing blood in and out of the VAD. With respect to the valve shown in FIG. 2, the blood would flow upward from an inlet port 36 to an outlet port 38. The valving function is provided by two symmetrical flaps 40 that are attached to the interior surface of the housing 32 by means of struts 42. A support ring 44 provides a base with an interior opening 46 (seen in FIG. 5) and a conforming seat 48 (also seen in FIG. 5) where the flaps 40 rest when the valve is closed.

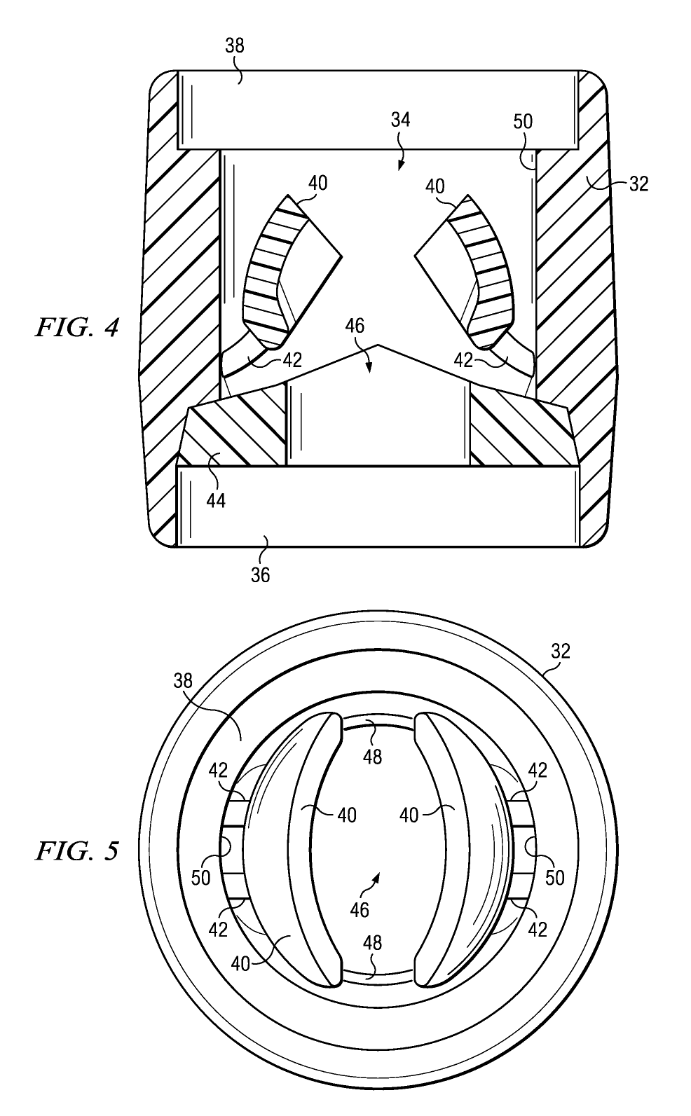

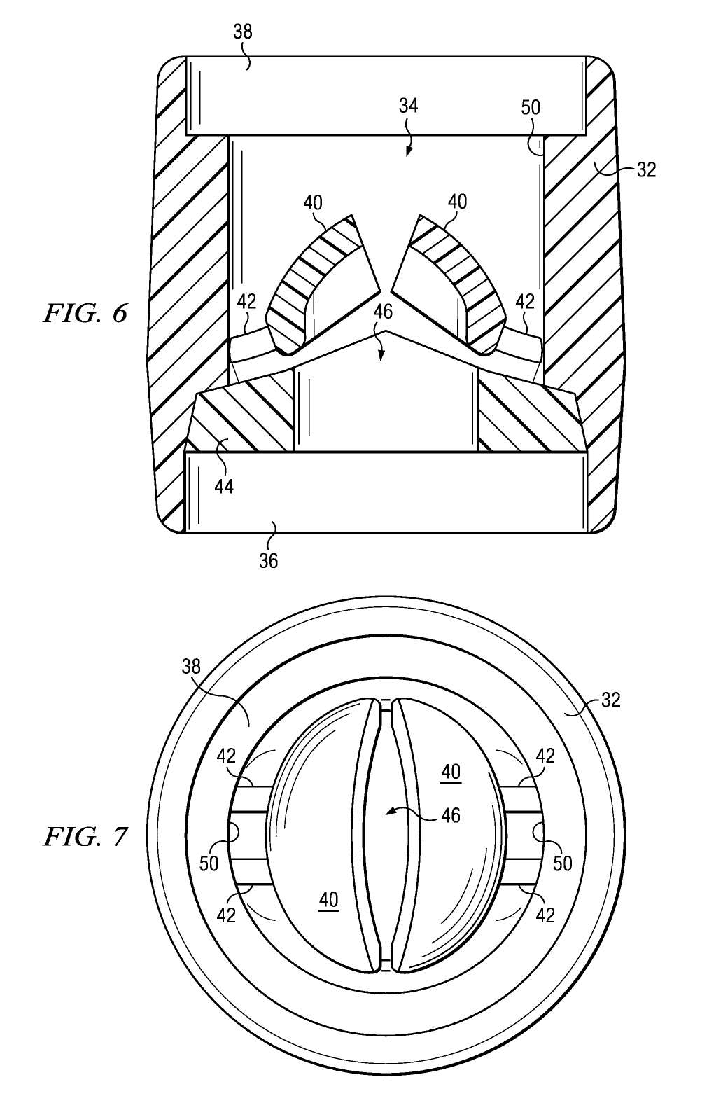

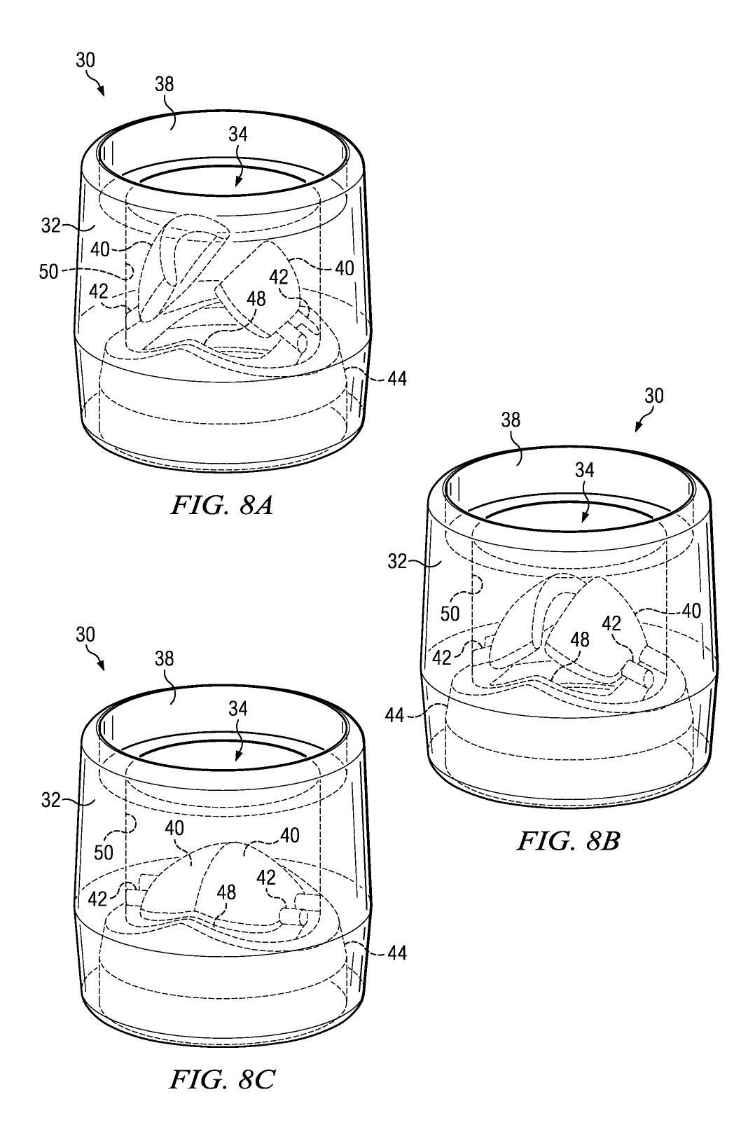

For ease of description, the terms up and down are used with reference to the figures in describing the function of the valve 30, it being understood that the actual position of the valve components and the direction of flow would in fact depend on the placement of the valve in the VAD. As seen in the top view of FIG. 3, where the valve is in closed position (i.e., the flaps 40 are seated in the seat 48 of the ring 44), the struts 42 are connected to the flaps 40 and the wall of the housing 32 in a manner that leaves open spaces all around them to allow blood flow between the flaps and the wall when the valve is open. As shown in FIGS. 4 and 5, where the valve is in fully open position, when the VAD pump exerts an upward pressure differential on the valve (either in suction or compression), the flaps open and blood flows upward through the opening 46 of the ring 44. As also illustrated by the intermediate flap position shown in FIGS. 6 and 7, the struts 42 define open spaces all around them at all times during the cycle of operation, so that blood may flow freely around them and wash out any stagnant cells residing on the surface of the various valve components. FIG. 8 shows three stages of the cycle of operation (from fully open to fully closed) in perspective view.

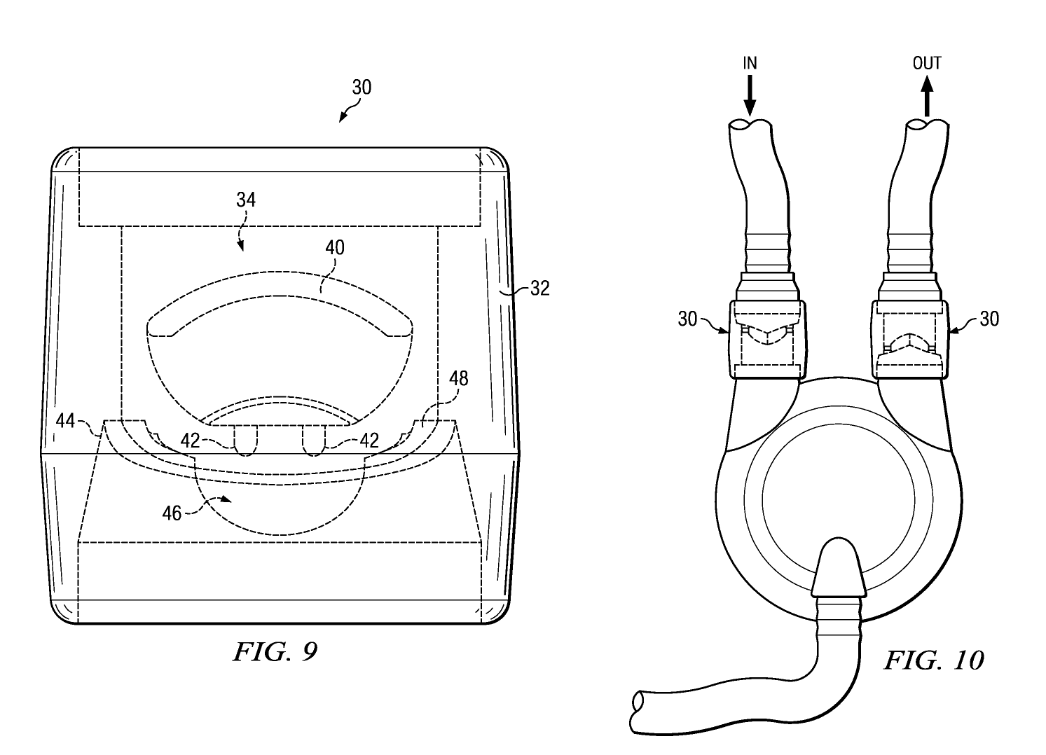

FIG. 8 and the partially cut-out elevational view of FIG. 9 illustrate the preferred configuration of the flap 40 of the invention. It consists essentially of a half dome (i.e., one half of a concave structural element that resembles the hollow upper half or upper portion of a sphere) sized to mesh with a corresponding portion of the seat 48 of the support ring 44 of the valve and with the other flap 40 covering the opposite side of the seat 48. Both flaps are preferably identical and each is preferably connected to the interior wall 50 of the valve housing (see FIGS. 2 and 3) by means of two struts 42. As a novel element of the present invention, these struts are tensioned so as to create a bias toward the closed position of the valve. As a result, when the flaps are pushed open by the pressure differential produced by the VAD pump, the struts' tension and their downward bias increases progressively as the flaps open. This condition creates a separate pressure differential between the bottom and the edges of the flaps that promotes flow all around them. In addition, inasmuch as this tension decreases the pressure drop across the flaps, the flow around them tends to be less turbulent with the net result that thrombus is greatly reduced with respect to conventional hinge designs.

As illustrated in the figures and well understood by one skilled in the art, the exact shape of the flaps 40 is not as crucial to the invention as the strut tensioning and the configuration of the strut attachment to the housing, so long as the struts are appropriately dimensioned to conform with and mesh well with each other and the seat of the valve to properly prevent back-flow in their closed position. As such, it is anticipated that the invention could be practiced with comparably advantageous results using three equal flaps, each designed to cover one third of the opening in the valve. The use of a single flap, while possible, would introduce undesirable flow asymmetries that could promote clotting and therefore it is not recommended. Similarly, a different number of struts could be used for each flap, though not recommended because a single strut might cause uneven flap motion and more than two struts would be an unnecessary complication.

The valve of the invention is currently being tested for marketing by SynCardia Systems, Inc., of Tucson, Ariz. The valve is injection-molded in several polyurethane parts (the housing, the struts and flaps, and the ring) that are then assembled and glued together into a single valve unit. While polyurethane is the preferred material, the valve could be made as well with other synthetic materials, such as silicone rubber, a thermoplastic elastomer (TPE), or polyvinyl chloride (PVC).

The SynCardia valve was tested in a conventional VAD, such as illustrated in FIG. 10, to assess its effectiveness in reducing thrombus and, accordingly, the need for anticoagulants. In a comparative animal study with a VAD that is known to begin showing evidence of clotting within about seven days of continuous use without anticoagulants (the norm in prior-art devices), the valves of the invention showed no sign of thrombus after 30 days of continuous operation. Therefore, it is clear that the structural design of the strut of the invention combined with its tensioned bias toward a closed position produces a significant advantage over all prior-art VAD valves. As a consequence, it is anticipated that the valve of the invention will enable the long-term use of VADs with a greatly diminished need for anticoagulants.

While the invention has been shown and described herein with reference to what is believed to be the most practical embodiment, it is recognized that departures can be made within the scope of the invention. For example, the struts of the invention may be attached to the housing with glue or be formed as a single structure. Therefore, the invention is not to be limited to the disclosed details, but is intended to embrace all equivalent structures and methods.

I claim:

1. A valve for an artificial blood pump comprising:
a valve seat defining a flow passage in a housing;
at least one flap adapted for meshing with the valve seat when the valve is closed; and
at least one flexible strut providing a hinge for the flap whereby the flap can move from a closed position to an open position and vice versa;
wherein said strut is tensioned to provide a bias toward said closed position of the flap, and said flap and strut define passages for fluid flow between the flap and the strut when the valve is open.

2. The valve of claim 1, wherein said flexible strut is attached to the housing.

3. The valve of claim 1, wherein said valve is made of polyurethane.

4. The valve of claim 1, wherein said valve is made of silicone rubber.

5. The valve of claim 1, wherein said valve is made of a thermoplastic elastomer.

6. The valve of claim 1, wherein said valve is made of polyvinyl chloride.

7. The valve of claim 1, wherein the valve includes two substantially identical flaps, each flap being adapted for meshing with the valve seat and with the other flap when the valve is closed.

8. The valve of claim 7, wherein each of said flaps comprises a portion shaped like a half dome.

9. The valve of claim 7, wherein each flap is attached to two substantially identical flexible struts, said struts providing a hinge for the flap whereby the flap can move from a closed position to an open position and vice versa; and wherein said struts are tensioned to provide a bias toward said closed position of the flap and said flap and struts define passages for fluid flow between the flap and the struts when the valve is open.

10. The valve of claim 9, wherein said flexible struts are attached to the housing.

11. The valve of claim 9, wherein each of said flaps comprises a portion shaped like a half dome.

12. The valve of claim 9, wherein said valve is made of polyurethane.

13. The valve of claim 9, wherein said valve is made of silicone rubber.

14. The valve of claim 9, wherein said valve is made of a thermoplastic elastomer.

15. The valve of claim 9, wherein said valve is made of polyvinyl chloride.

16. A ventricular assist device comprising:
a pump chamber with an inlet port, an outlet port, and a mechanism for creating suction at the inlet port and pressure at the outlet port;
an inlet valve allowing flow into the chamber when said mechanism creates said suction at the inlet port and preventing backflow out of the chamber when the mechanism creates said pressure at the outlet port; and
an outlet valve preventing backflow into the chamber when said mechanism creates said suction at the inlet port and allowing flow out of the chamber when the mechanism creates said pressure at the outlet port;
wherein each of said inlet and outlet valves includes:
a valve seat defining a flow passage in a housing;
two substantially identical flaps, each flap being adapted for meshing with the valve seat and with the other flap when the valve is closed; and
two substantially identical flexible struts attached to each flap, said struts providing a hinge for the flap whereby the flap can move from a closed position to an open position and vice versa;
wherein the struts are tensioned to provide a bias toward said closed position of the flap and said flap and struts define passages for fluid flow between the flap and the struts when the valve is open.

17. The valve of claim 16, wherein each of said flaps comprises a portion shaped like a half dome.

18. The valve of claim 16, wherein said flexible struts are attached to the housing.

19. The valve of claim 16, wherein said valve is made of polyurethane.

20. The valve of claim 16, wherein each of said flaps comprises a portion shaped like a half dome, said flexible struts are attached to the housing, and the valve is made of polyurethane.
